(19) United States
(12) Patent Application Publication
(10) Pub. No.: US 2023/0301810 A1
(43) Pub. Date: Sep. 28, 2023

(54) METHODS FOR SHORTENING A DUODENUM OF A SUBJECT

(71) Applicant: Digma Medical Ltd., Giv'at Schmuel (IL)

(72) Inventors: Shlomit CHAPPEL-RAM, Ramat-Hasharon (IL); Avia HERSCHKOVITZ, Ness-Ziona (IL)

(21) Appl. No.: 18/124,376

(22) Filed: Mar. 21, 2023

Related U.S. Application Data
(60) Provisional application No. 63/324,090, filed on Mar. 27, 2022.

(51) Int. Cl. A61F 5/00

(52) U.S. Cl. CPC A61F 5/0076

(57) ABSTRACT

A method for shortening a duodenum of a subject, the method including: manipulating a first surface of the duodenum wall such that a first portion of the first surface of the duodenum wall is layered onto a second portion of the first surface of the duodenum wall, thereby creating at least one fold in the first surface of the duodenum wall, and securing, utilizing at least one implant, the position of the at least one fold in relation to a surrounding surface of the duodenum wall, thereby shortening the duodenum of the subject.

### CROSS REFERENCE TO RELATED APPLICATIONS

This application claims the benefit of priority of U.S. Provisional Pat. Application No. 63/324,090, filed Mar. 27, 2022, the contents of which are all incorporated herein by reference in their entirety.

### TECHNICAL FIELD

The present disclosure relates generally to methods for shortening a duodenum of a subject.

### BACKGROUND

Today, bariatric surgery that excludes the passage of food through the duodenum is the most effective treatment of Type II Diabetes. About 90% of diabetic patients who underwent a R&Y gastric bypass or BPD were relieved of their symptoms. These, however, are surgical procedures conducted on only 0.1% of the patient population. Since the duodenum plays a unique and important role in glycemic control and when bypassed the effects are enhanced, there have been multiple attempts to induce similar effects using different instrumental interventions such as GI Dynamic’s EndoBarrier Duodenal-jejunal bypass liner, Fractyl Health’s Revita system for Duodenal Mucosal Resurfacing, and Digma Medical’s minimally invasive therapy, that involves a laser-based ablation treatment of the Duodenum, the Endoscopic Glycemic Management (EGM) procedure which is interrupting metabolic signaling from the gut, reducing insulin resistance and improving glycemic control, and effectively avoiding nutrients from passing through the duodenum, thus, allowing the body to once again naturally control glucose levels.

### SUMMARY

According to some embodiments there is provided herein methods for modulation of the duodenal tissue inter alia modulation of neural components contained therein and/or interacting with such as, but not limited to, neural activity and/or sensing and/or changing of the timing of the meal passage.

According to some embodiments, the method may be configured to shorten the duodenal active area, thereby making its internal surface minimally exposed. According to some embodiments, the procedure could be controlled, per specific anatomy and the needs and conditions of the subject.

According to some embodiments, there is provided a method for shortening a duodenum of a subject, the method comprising: manipulating a first surface of the duodenum wall such that a first portion of the first surface of the duodenum wall is layered onto a second portion of the first surface of the duodenum wall, thereby creating at least one fold in the first surface of the duodenum wall; and securing, utilizing at least one implant, the position of the at least one fold in relation to a surrounding surface of the duodenum wall, thereby shortening the duodenum of the subject.

According to some embodiments, the first surface of the duodenum wall comprises at least a portion of an inner surface of the duodenum wall. According to some embodiments, the first surface of the duodenum wall comprises at least a portion of an outer surface of the duodenum wall.

According to some embodiments, the method further comprises controlling the tension of the duodenum wall by changing the size of the fold.

According to some embodiments, the method further comprises creating a plurality of folds. According to some embodiments, the at least two of the plurality of folds are abutting. According to some embodiments, the at least two of the plurality of folds are spaced apart.

According to some embodiments, the at least one fold extends circumferentially around the duodenum wall. According to some embodiments, at least two of the plurality of folds are parallel to each other.

According to some embodiments, the at least one fold is essentially parallel to a longitudinal axis of the duodenum. According to some embodiments, the at least one fold is essentially perpendicular to a longitudinal of the duodenum.

According to some embodiments, the at least one implant is composed of an absorbable material configured to absorb into the tissue of the subject.

According to some embodiments, the at least one implant comprises a wire, and wherein securing the position of the at least one fold comprises suturing the duodenum wall using the wire.

According to some embodiments, the at least one implant comprises a biological glue. According to some embodiments, the securing of the position of the at least one fold comprises gluing the first surface of the duodenum wall to the second surface of the duodenum wall.

According to some embodiments, the at least one implant comprises a locking mechanism. According to some embodiments, the locking mechanism comprises two lockable portions. According to some embodiments, the securing of the position of the at least one fold comprises securing a first lockable portion of the locking mechanism to the first surface of the duodenum wall; securing a second lockable portion of the locking mechanism to the second surface of the duodenum wall; and locking the first lockable portion with the second lockable portion.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes. In the figures:

FIG. 1 shows a flow chart of the steps of a method for shortening a duodenum of a subject, in accordance with some embodiments of the present invention;

FIG. 2 shows a duodenum wall two folds, in accordance with some embodiments of the present invention;

FIG. 3A and FIG. 3B show schematic illustrations of exemplary shortened duodenums having a plurality of folds, in accordance with some embodiments of the present invention;

FIG. 4 shows a schematic illustration of an exemplary shortened duodenum wall with one fold per each wall, in accordance with some embodiments of the present invention.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, duodenal shrinkage, or the shortening of the duodenum, may be created using a permanent implant left in duodenum area. According to some embodiments, duodenal shrinkage, or the shortening of the duodenum, may be created using a temporary implant implanted into the duodenum area. According to some embodiments, duodenal shrinkage may be achieved using a temporary adsorbable implant. According to some embodiments, the method may be a minimally invasive procedure. According to some embodiments, the duodenum shortening may be achieved by creating an accordion-like pleating shape of the duodenum wall, or in other words, duodenal plication. According to some embodiments, duodenum shortening may be achieved by rolling a redundant portion of the duodenum wall onto the active portion of the duodenum wall, thereby reducing the overall length of the duodenum.

Advantageously, the method described herein may enable controlling the length by which the duodenum is shortened. The method described herein can also be adjusted based on the anatomy, conditions, and needs of the subject prior and/or during the procedure for shortening the duodenum.

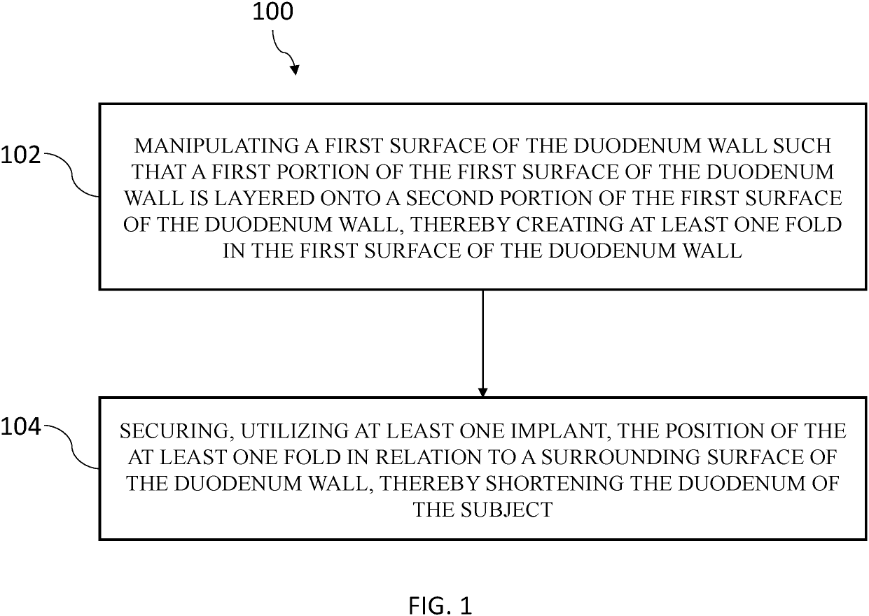

Reference is made to FIG. 1, which shows a flow chart of the steps of a method for shortening a duodenum of a subject, in accordance with some embodiments of the present invention. According to some embodiments there is provided a method for shortening a duodenum of a subject, such as the method 100 as depicted in FIG. 1.

According to some embodiments, at step 102, the method 100 may include manipulating a first surface of the duodenum wall such that a first portion of the first surface of the duodenum wall is layered onto a second portion, or brought into proximity to a second portion of the first surface of the duodenum wall, thereby creating at least one fold in the first surface of the duodenum wall. According to some embodiments, at step 104, the method 100 may include securing, utilizing at least one implant (permanent or temporary), the position of the at least one fold in relation to a surrounding surface of the duodenum wall, thereby shortening the duodenum of the subject.

According to some embodiments, the method may include inserting a medical tool into the duodenum of a subject. According to some embodiments, the medical tool may include an imaging device configured to enable a user to view the duodenum of a subject. According to some embodiments, the medical tool may include a manipulatable tip configured to support an implant. According to some embodiments, the method may include positioning the implant onto the medical tool. According to some embodiments, the method may include manipulating the orientation and/or the position of the implant by manipulating the tip of the medical tool.

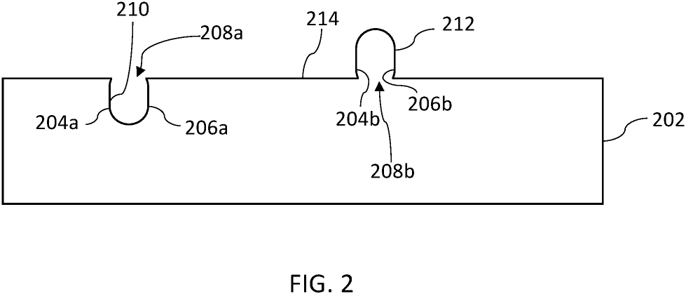

Reference is made to FIG. 2 which shows a duodenum wall two folds, in accordance with some embodiments of the present invention.

According to some embodiments, the method may include manipulating the duodenum of the subject, or the duodenum wall of the subject, such as, for example the duodenum wall 202 as depicted in FIG. 2. According to some embodiments, the method may include manipulating the duodenum of the subject using the medical tool and/or the implant at the tip thereof. According to some embodiments, the method may include manipulating a first surface of the duodenum wall such that a first portion 204a/204b of the first surface of the duodenum wall is layered onto a second portion 206a/206b of the first surface of the duodenum wall 202. According to some embodiments, the method may include creating at least one fold 208a/208b in the first surface of the duodenum wall 202.

According to some embodiments, the first surface of the duodenum wall comprises at least a portion of an outer surface 210 of the duodenum wall. According to some embodiments, the method may include creating at least one fold 208a such that the first portion 204a of the outer surface 210 of the duodenum wall is layered onto the second portion 206a of the outer surface 210 of the duodenum wall. According to some embodiments, the first surface of the duodenum wall comprises at least a portion of an inner surface 212 of the duodenum wall. According to some embodiments, the method may include creating at least one fold 208b such that the first portion 204b of the inner surface 212 of the duodenum wall is layered onto the second portion 206b of the inner surface 212 of the duodenum wall.

According to some embodiments, the method may include controlling the tension of the duodenum wall by changing the size of the fold. According to some embodiments, the method may include identifying the required tension of the duodenum for the individual subject. According to some embodiments, the method may include identifying the required size of the fold for the individual subject. According to some embodiments, the size of the fold may include the length, width, and/or area of the layered first portion and second portions of the duodenum wall. According to some embodiments, for an operation including a plurality of folds, the size of the fold may include the total length, width, and/or area of the plurality of folds (or in other words, the total reduction length, width, and/or area of the duodenum wall).

According to some embodiments, the method may include applying a predetermined amount of force, using the medical tool and/or the implant, thereby achieving a specified size of the fold. According to some embodiments, the method may include identifying and/or calculating the predetermined amount of force required to create a specific fold, based on the specific duodenum of the subject (or in other words, personalize the size of the fold and/or the force required based on the specific duodenum of the subject). According to some embodiments, the method may include controlling the orientation and/or direction in which the implant is placed. According to some embodiments, the method may include identifying and/or calculating the required orientation and/or position of the implant for creating a specific fold in the duodenum of the subject.

According to some embodiments, the method may include creating a plurality of folds in the duodenum wall of the subject. According to some embodiments, the method may include identifying the required location for the plurality of folds. According to some embodiments, the method may include creating a plurality of fold of which at least two folds are abutting (or in other words, creating pleats). According to some embodiments, the method may include creating a plurality of fold of which at least two are spaced apart (or in other words, there is a duodenum wall that has not been folded between the at least two folds, such as, for example, the straight portion 214 of the duodenum wall 202 between folds 208a and 208b). According to some embodiments, the method may include creating at least one fold which extends circumferentially around the duodenum wall. According to some embodiments, the method may include creating at least one fold which extends helically around the duodenum wall. According to some embodiments, the fold may be (essentially) parallel to a longitudinal axis of the duodenum wall. According to some embodiments, the fold may be (essentially) perpendicular to the longitudinal axis of the duodenum wall. According to some embodiments, two or more folds may be parallel to each other. According to some embodiments, two or more folds may cross each other.

According to some embodiments, at step 104, the method 100 may include securing, utilizing at least one implant, the position of the at least one fold in relation to a surrounding surface of the duodenum wall, thereby shortening the duodenum of the subject.

According to some embodiments, the implant may be composed of an absorbable material configured to absorb into the tissue of the subject. According to some embodiments, the implant may be composed of a biodegradable material configured to absorb into the duodenum of the subject.

According to some embodiments, the implant may include a wire, or a suture. According to some embodiments, the wire may be composed of a compatible and/or adsorbable material, such as, for example, a compatible Polyethylene wire, a compatible thin metal wire, and the like.

According to some embodiments, securing the position of the at least one fold may include suturing the duodenum wall using the wire. According to some embodiments, the method may include sewing (or suturing) the duodenum wall into the at least one fold. According to some embodiments, the method may include sewing at least one fold starting from the proximal side of the duodenum wall. According to some embodiments, the method may include sewing the at least one fold starting from the distal side of the duodenum wall. According to some embodiments, the method may include sewing the at least one fold by starting from the proximal and/or distal side of the duodenum wall and then moving to the other side of the wall continuously. According to some embodiments, the method may include sewing each side of the duodenum wall separately.

According to some embodiments, the method may include sewing the ‘redundant’ parts of the duodenum parts into extramural positioning which can then be shortened. According to some embodiments, the method may include shortening the sewed extramural duodenum parts. According to some embodiments, the method may include removing the extramural duodenal parts, thereby affixing the anatomical alteration.

According to some embodiments, the implant may include a biological glue. According to some embodiments, the medical tool may include a biological glue dispenser configured to eject the biological glue. According to some embodiments, securing the position of the at least one fold may include gluing the first surface of the duodenum wall to the second surface of the duodenum wall. According to some embodiments, the biological glue may include, for example, any one or more of Fibrin, Hydrogel, Protein, and the like. According to some embodiments, the biological glue may be in the form of an aerosol, a liquid, and/or a gel material. According to some embodiments, the method may include applying the biological glue with a wire or suture. According to some embodiments, the method may include applying the biological glue without a wire or suture. According to some embodiments, the biological glue may be configured to transform into a durable, elastic and/or protective layer configured to conform to tissue and imitate its natural properties.

According to some embodiments, the biological glue may require chemical surface modification of the duodenum wall. According to some embodiments, the method may include applying the required chemical surface modifications to the duodenum wall prior to applying the biological glue. According to some embodiments, the biological glue may not need chemical surface modification of the duodenum wall prior to the application thereof.

According to some embodiments, the implant may include a locking mechanism. According to some embodiments, the locking mechanism may include two lockable portions. According to some embodiments, each of the two lockable portions may be configured to couple (or adhere) to the wall of the duodenum of the subject. According to some embodiments, the method may include securing the position of the at least one fold by securing a first lockable portion of the locking mechanism to the first surface of the duodenum wall. According to some embodiments, the method may include securing the position of the at least one fold by securing a second lockable portion of the locking mechanism to the second surface of the duodenum wall. According to some embodiments, the method may include locking the first lockable portion with the second lockable portion.

According to some embodiments, securing any one of the first lockable portion and/or the second lockable portion to the surface of the duodenum wall may include using wire, such as the wire as described in greater detail elsewhere herein. securing any one of the first lockable portion and/or the second lockable portion to the surface of the duodenum wall may include applying biological glue, such as the biological glue as described in greater detail elsewhere herein.

According to some embodiments, the first lockable portion and the second lockable portion may include one or more of hooks, geometrically compatible shapes, adhesives, loops, and the like. According to some embodiments, the locking mechanism may include velcro (or in other words, may include any one or more of hooks and loop fasteners on either one of the first lockable portion and the second lockable portion). For example, the locking mechanism may include a first strip including hooks, wherein the strip is configured to be attached to the first portion of the duodenum wall, and a second stipe including small loops, wherein the second strip is configured to be adhered to the second portion of the duodenum wall. According to some embodiments, the first and second strips may be configured such that when the two strips are pressed together, the hooks will catch the loops, thereby fastening the first portion and the second portion of the duodenum wall and securing the fold.

According to some embodiments, the method may be devoid of ablation. According to some embodiments, the method may be devoid of the use of lasers.

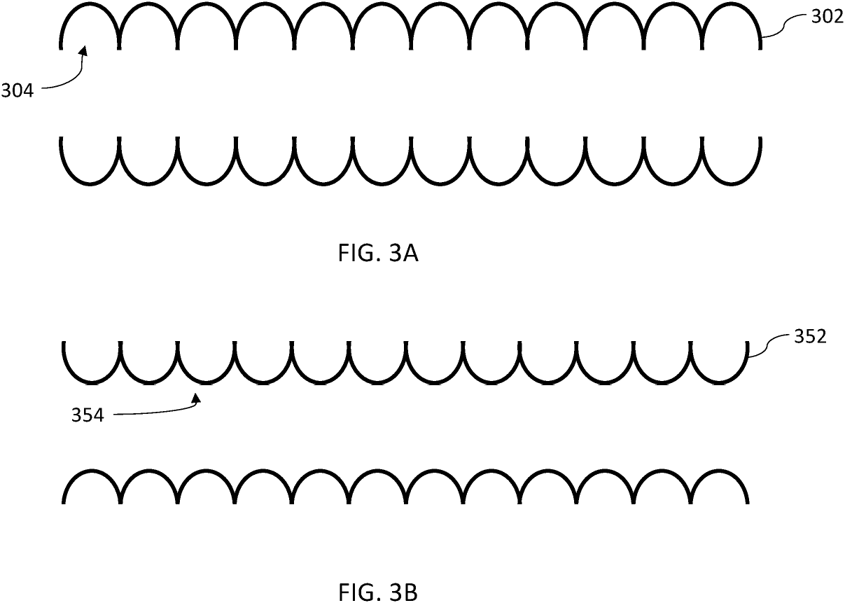

Reference is made to FIG. 3A and FIG. 3B, which show schematic illustrations of exemplary shortened duodenums having a plurality of folds, in accordance with some embodiments of the present invention.

According to some embodiments, such as depicted in FIG. 3A and FIG. 3B, the duodenum may be shortened by creating a plurality of folds, or pleats, in the duodenum wall of the subject. According to some embodiments, the method may include creating a plurality of pleats in the duodenum wall 302 of the subject, wherein the folds 304 are folded outwards, such as depicted in FIG. 3A. According to some embodiments, the method may include creating a plurality of pleats in the duodenum wall 352 of the subject, wherein the folds 354 are folded inwards, such as depicted in FIG. 3B.

According to some embodiments, the method may include plication of the duodenum wall, or in other words, shaping the duodenum wall in an accordion-like shape, thereby shortening the duodenum wall. According to some embodiments, the directional orientation of the folds may be controlled. According to some embodiments, the method may include controlling the tissue folds’ tightness. According to some embodiments, the method may include controlling the tissue folds’ tightness controlling the fold size, the size or direction of the increments of the duodenum wall being folded, and the applied force.

According to some embodiments, the method may include plication of the duodenum wall using a wire suture. According to some embodiments, the method may include, using a wire, lateral plane suturing of the duodenum walls to create the accordion folds/duodenal plication. According to some embodiments, the method may include sewing the duodenum wall in one or more different directions. According to some embodiments, the contraction of the wire may be dependent on the direction of the suturing. According to some embodiments, the method may include implementing one contraction point for opposing sides of the duodenum wall simultaneously. According to some embodiments, the method may include having one contraction point for each opposing side of the duodenum wall individually. According to some embodiments, and as described in greater detail elsewhere herein, the sewing wire may be composed of an adsorbable material, a Polyethylene wire, a compatible metal wire, and the like.

According to some embodiments, the method may include plication of the duodenum wall using biological glue, such as the biological glue described in greater detail elsewhere herein. According to some embodiments, the method may include using a biological glue for creating the accordion folds. According to some embodiments, the biological glue may be used with or without the use of a suture (or wire).

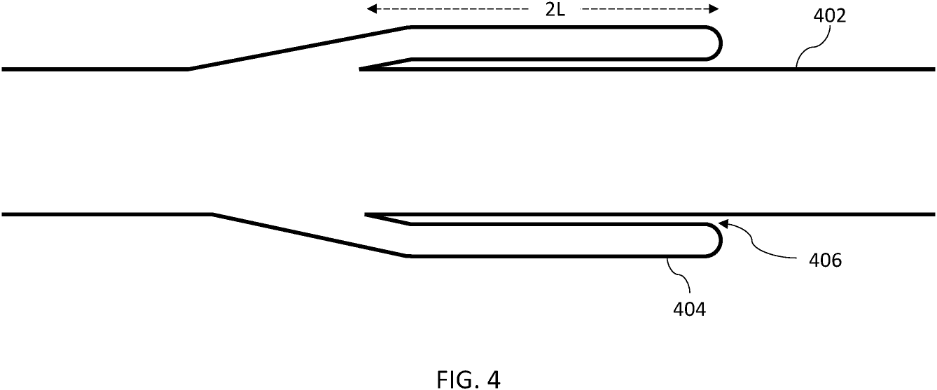

Reference is made to FIG. 4, which shows a schematic illustration of an exemplary shortened duodenum wall with one fold, in accordance with some embodiments of the present invention.

According to some embodiments, the method may include creating a fold in the form of a rolled sleeve. According to some embodiments, the rolled sleeve shape of the fold 404 may form a sheath surrounding the duodenum 402 of the subject. According to some embodiments, the method may include securing the fold 404 by abutting the first and second portions of the outer wall 406 of the duodenum, such as depicted in FIG. 4.

According to some embodiments, the method may include identifying the redundant portion of the duodenum wall. According to some embodiments, the length L of the redundant portion may vary depending on subject’s anatomy and conditions. According to some embodiments, the method may include folding the redundant portion by rolling up the redundant portion onto the active duodenum wall’s external surface (such as surface 406).

According to some embodiments, the method may include securing the rolled portion of the duodenum wall using a wire, such as the wire described in greater detail elsewhere herein. According to some embodiments, the method may include sewing the redundant portion such that the position of the redundant portion is extramurally positioned in relation to the duodenum. According to some embodiments, the method may include sewing the redundant portion to the outer duodenum wall, thereby leaving the redundant portion folded but still active in case there will be a need to extend the functioning duodenum area for specific patient following procedure. According to some embodiments, the method may include permanently removing the folded redundant portion (or in other words, the extramural duodenal tissue), thereby affixing the anatomical alteration of the duodenum and shortening thereof.

According to some embodiments, the method may include securing the rolled portion of the duodenum wall using a biological glue, such as the biological glue described in greater detail elsewhere herein. According to some embodiments, the method may include using the biological glue to adhere the rolled redundant portion to the outer surface of the duodenum wall. According to some embodiments, the method may include securing the rolled portion of the duodenum wall using a lockable mechanism. According to some embodiments, the method may include securing the rolled portion of the duodenum wall using Velcro (or hooks and loop fasteners). According to some embodiments, the method may include coupling a first strip to the outer surface of the duodenum wall (the external portion of the active area of the duodenum). According to some embodiments, the method may include coupling a second strip, configured to lock with the first strip, to the folded (or rolled) redundant portion of the duodenum. According to some embodiments, the first and/or second strips may include a loops and hooks mechanism configured to lock when pressed together. According to some embodiments, the method may include pressing the folded (or rolled) portion and the outer surface of the duodenum wall together, thereby fastening (or locking) the folded (or rolled) portion to the outer surface of the duodenum wall.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for shortening a duodenum of a subject, the method comprising:
manipulating a first surface of the duodenum wall such that a first portion of the first surface of the duodenum wall is layered onto a second portion of the first surface of the duodenum wall, thereby creating at least one fold in the first surface of the duodenum wall; and
securing, utilizing at least one implant, the position of the at least one fold in relation to a surrounding surface of the duodenum wall, thereby shortening the duodenum of the subject.

2. The method according to claim 1, wherein the first surface of the duodenum wall comprises at least a portion of an inner surface of the duodenum wall.

3. The method according to claim 1, wherein the first surface of the duodenum wall comprises at least a portion of an outer surface of the duodenum wall.

4. The method according to claim 1, further comprising controlling the tension of the duodenum wall by changing the size of the fold.

5. The method according to claim 1, further comprising creating a plurality of folds.

6. The method according to claim 5, wherein at least two of the plurality of folds are abutting.

7. The method according to claim 5, wherein at least two of the plurality of folds are spaced apart.

8. The method according to claim 1, wherein the at least one fold extends circumferentially around the duodenum wall.

9. The method according to claim 8, wherein at least two of the plurality of folds are parallel to each other.

10. The method according to claim 1, wherein the at least one fold is essentially parallel to a longitudinal axis of the duodenum.

11. The method according to claim 1, wherein the at least one fold is essentially perpendicular to a longitudinal axis of the duodenum.

12. The method according to claim 1, wherein the at least one implant is composed of an absorbable material configured to absorb into the tissue of the subject.

13. The method according to claim 1, wherein the at least one implant comprises a wire, and wherein securing the position of the at least one fold comprises suturing the duodenum wall using the wire.

14. The method according to claim 1, wherein the at least one implant comprises a biological glue, and wherein securing the position of the at least one fold comprises gluing the first surface of the duodenum wall to the second surface of the duodenum wall.

15. The method according to claim 1, wherein the at least one implant comprises a locking mechanism, the locking mechanism comprising two lockable portions, and wherein securing the position of the at least one fold comprises:
securing a first lockable portion of the locking mechanism to the first surface of the duodenum wall;
securing a second lockable portion of the locking mechanism to the second surface of the duodenum wall; and
locking the first lockable portion with the second lockable portion.
